(19) United States
(12) Patent Application Publication
(10) Pub. No.: US 2026/0272318 A1
(43) Pub. Date: Sep. 17, 2026

(54) THERMAL STRESS MONITORING SYSTEM AND METHOD THEREOF

(71) Applicant: Vivonics, Inc., Bedford, MA (US)

(72) Inventor: Michele Pierro, Westford, MA (US)

(21) Appl. No.: 19/090,656

(22) Filed: Mar. 26, 2025

Related U.S. Application Data
(60) Provisional application No. 63/570,865, filed on Mar. 28, 2024.

(51) Int. Cl. A61B 5/0295; A61B 5/00

(52) U.S. Cl. CPC A61B 5/0295; A61B 5/6823; A61B 2503/10; A61B 2503/20

(57) ABSTRACT

A thermal stress monitoring system includes at least one wearable sensor adapted to be placed on a human subject. The at least one wearable sensor is sensitive to changes in spontaneous blood volume oscillations and is configured to generate output signals. A processing subsystem receives the output signals and measures baseline spontaneous blood volume oscillations at a time when the user is not exposed to a thermal stress environment and subsequently measures the exposed spontaneous blood volume oscillations at a time when the user is exposed to the thermal stress environment and determines a thermal stress injury based on changes in the measured exposed spontaneous blood volume oscillations from the measured baseline spontaneous blood volume oscillations.

### RELATED APPLICATIONS

This application claims benefit of and priority to U.S. Provisional Application Ser. No. 63/570,865 filed Mar. 28, 2024, under 35 U.S.C. §§ 119, 120, 363, 365, and 37 C.F.R. § 1.55 and § 1.78, which is incorporated herein by this reference.

### GOVERNMENT RIGHTS

This invention was made with U.S. Government support under Contract No. HT9425-23-1-0874 awarded by the U.S. Army. The Government has certain rights in the invention.

### FIELD OF THE INVENTION

This invention relates to a thermal stress monitoring system and method thereof

### BACKGROUND OF THE INVENTION

Thermal stress may include frostbite and related cold injuries, heat strain, heat stroke, heat exhaustion and the like. Thermal stress injuries may affect military personnel, outdoor sports athletes, construction and utility workers, homeless or displaced persons, and the like.

Frostbite typically affects the hands, fingers, toes, and feet but can also occur on the ears, nose, and cheeks. Like any condition that increases localized heat loss or decreases heat production, frostbite generally develops within minutes to hours depending on circumstances and risk factors. Diagnosis is generally made on clinical grounds, based on the context of the injury, signs, and symptoms. Conventional imaging techniques such as thermography, Doppler, radiography, angiography, and the like, can help to determine the extent of tissue involvement and to predict response to therapy and long-term tissue viability. At times, invasive sensory measurements utilizing sterile needles are used to test proprioception. None of these conventional imaging modalities or invasive techniques are sufficiently portable and able to operate continuously and therefore are not suitable to be performed in real-time in the field during training or other activities in austere environments. Additionally, these conventional imaging modalities or invasive techniques may not provide any relevant information with regard to frostbite initiation, a capability that is essential to reduce the negative impact of frostbite on the operational performance and readiness of those affected by frostbite.

Hypothermia is a significant drop in body temperature when the body's heat loss exceeds its production resulting in the body not being able to maintain its normal core body temperature.

Frostnip happens before frostbite and occurs when the superficial skin cools to below 50° F. Frostnip typically occurs on the hands, fingers, toes, and feet but can also occur on the ears, nose, and cheeks and typically does not cause permanent damage, but may cause pain or numbness.

Chilblain, a non-freezing cold related injury, is related to extended exposure to thermal stress environments and wet conditions and typically occurs on the hands and feet.

Immersion foot typically occurs with prolonged exposure to a cold and wet environment with temperatures in the range of about 32° F. to 65° F. It usually affects the soft tissues, including nerves and blood vessels, due to an inflammatory response leads to high levels of extracellular fluid.

Exposure to high levels of heat and humidity may cause heat strain, heat stroke, heat cramps, heat exhaustion, and the like.

Conventional techniques to detect thermal stress on a human subject may include taking oral and rectal temperatures, neurological assessments, and visual inspection of the victim which may be cumbersome and inaccurate. The conventional imaging modalities or invasive techniques discussed above may not provide any relevant information with regard hypothermia, frostnip, chilblain, immersion foot heat stroke, heat stain, and similar thermal stress injuries.

### SUMMARY OF THE INVENTION

In one aspect a thermal stress monitoring system is featured. The system includes at least one wearable sensor adapted to be placed on a human subject. The at least one wearable sensor is preferably sensitive to changes in spontaneous blood volume oscillations and preferably generates output signals. A processing subsystem preferably receives the output signals and measures baseline spontaneous blood volume oscillations at a time when the user is not exposed to a thermal stress environment and subsequently measure the exposed spontaneous blood volume oscillations at a time when the user is exposed to the thermal stress environment and determine a thermal stress injury based on changes in the measured exposed spontaneous blood volume oscillations from the measured baseline spontaneous blood volume oscillations.

In one embodiment, the changes in the measured exposed spontaneous blood volume oscillations from the measured baseline spontaneous blood volume oscillations may be determined by evaluating the difference between the measured exposed spontaneous blood volume oscillations and the measured baseline spontaneous blood volume oscillations. The difference between the measured exposed spontaneous blood volume oscillations and the measured baseline spontaneous blood volume oscillations may include one or more comparisons that utilize time domain differences, frequency domain differences, or both. The at least one wearable sensor may include at least one photoplethysmography (PPG) sensor, at least one bioimpedance analysis (BIA) sensor, and/or at least one ballistocardiography (BCG) sensor. The at least one wearable sensor may include at least one light source configured to emit light at one or more predetermined wavelengths associated with spontaneous blood volume oscillations into tissue of the human subject and at least one detector configured to detect reflected light at the one or more predetermined wavelengths associated with the spontaneous blood volume oscillations and generate the output signals.

In another aspect, a thermal stress monitoring system is featured. The system preferably includes at least one peripheral wearable sensor adapted to be placed on a peripheral area of a user. The peripheral wearable sensor preferably includes at least one peripheral light source which preferably emits light at one or more predetermined wavelengths associated with spontaneous blood volume oscillations into tissue of a human subject and at least one peripheral detector which preferably detects reflected light at the one or more predetermined wavelengths associated with the spontaneous blood volume oscillations and generate peripheral output signals. At least one baseline wearable sensor is preferably adapted to be placed on a core area of a user. The baseline wearable sensor preferably includes at least one baseline light source which preferably emits light at one or more predetermined wavelengths associated with spontaneous blood volume oscillations into tissue of a human subject and at least one baseline detector which preferably detects reflected light at the one or more predetermined wavelengths associated with the spontaneous blood volume oscillations and generate baseline output signals. A processing subsystem preferably receives the baseline output signals and measures the baseline spontaneous blood volume oscillations and preferably receives the peripheral output signals and preferably measure the peripheral spontaneous blood volume oscillations at a time when the user is exposed to a thermal stress environment and determine a thermal stress injury based on changes in the measured peripheral blood volume oscillations from the measured baseline spontaneous blood volume oscillations.

In one embodiment, the changes in the measured peripheral spontaneous blood volume oscillations from the measured baseline spontaneous blood volume oscillations may be determined by evaluating the difference between the measured peripheral spontaneous blood volume oscillations and the measured baseline spontaneous blood volume oscillations. The difference between the measured peripheral spontaneous blood volume oscillations and the measured baseline spontaneous blood volume oscillations may include one or more comparisons that utilize time domain differences, frequency domain differences, or both. The at least one wearable baseline sensor may be adapted to be placed on a core area of the user. The at least one wearable baseline sensor may generate at least one reference signal to be used by the processing subsystem to improve the accuracy of the determined thermal stress injury.

In yet another aspect, a system for detecting at least one condition that restricts perfusion of peripheral tissue is featured. The system preferably includes at least one wearable sensor adapted to be placed on a human subject. The at least one wearable sensor is preferably sensitive to changes in spontaneous blood volume oscillations and preferably generates output signals. A processing subsystem preferably receives the output signals and measures baseline spontaneous blood volume oscillations at a time when the user does not have at least one condition that restricts perfusion of peripheral tissue and subsequently measure conditional spontaneous blood volume oscillations at a time when the user has at least one condition that restricts perfusion of peripheral tissue and preferably determines at least one condition that restricts perfusion of peripheral tissue based on changes in the measured conditional spontaneous blood volume oscillations from the baseline spontaneous blood volume oscillations.

In one embodiment, the changes in the measured conditional spontaneous blood volume oscillations from the measured baseline spontaneous blood volume oscillations may be determined by evaluating the difference between the measured conditional spontaneous blood volume oscillations and the measured baseline spontaneous blood volume oscillations. The difference between measured conditional spontaneous blood volume oscillations and the measured baseline spontaneous blood volume oscillations may include one or more comparisons that utilize time domain differences, frequence domain differences, or both. The at least one wearable sensor may include photoplethysmography (PPG) sensor, at least one bioimpedance analysis (BIA) sensor, and/or at least one ballistocardiography (BCG) sensor The at least one wearable sensor may include at least one light source configured to emit light at one or more predetermined wavelengths associated with spontaneous blood volume oscillations into tissue of the human subject and at least one detector configured to detect reflected light at the one or more predetermined wavelengths associated with the spontaneous blood volume oscillations and generate the output signals.

In another aspect, a thermal stress monitoring method is featured. The method preferably includes measuring changes in spontaneous blood volume oscillations and generating output signals, responding to the output signals, and measuring baseline spontaneous blood volume oscillations at a time when the user is not exposed to a thermal stress environment and subsequently measuring the exposed spontaneous blood volume oscillations at a time when the user is exposed to the thermal stress environment and determining a thermal stress injury based on changes in the measured exposed spontaneous blood volume oscillations from the measured baseline spontaneous blood volume oscillations.

In one embodiment, the changes in the measured exposed spontaneous blood volume oscillations from the measured baseline spontaneous blood volume oscillations may be determined by evaluating the difference between the measured exposed spontaneous blood volume oscillations and the measured baseline spontaneous blood volume oscillations. The difference between the measured exposed spontaneous blood volume oscillations and the measured baseline spontaneous blood volume oscillations may include one or more comparisons that utilize time domain differences, frequency domain differences, or both. The method may include emitting light at one or more predetermined wavelengths associated with spontaneous blood volume oscillations into tissue of the human subject and detecting reflected light at the one or more predetermined wavelengths associated with the spontaneous blood volume oscillations and generating the output signals.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic view showing one example of the thermal stress monitoring system;

FIG. 2A shows an example of measured exposed spontaneous blood volume oscillations at a time when the user is exposed to a thermal stress environment and the measured baseline spontaneous blood volume oscillations at a time when user is not exposed to a thermal stress environment;

FIG. 2B shows an example of amplitudes change as measured by the processing subsystem shown in FIG. 1 compared to actual oscillation amplitudes;

FIG. 3 is a schematic view showing an example of the thermal stress monitoring system shown in FIG. 1 including a peripheral sensor;

FIG. 4A shows an example of measured peripheral spontaneous blood volume oscillations at a time when the user is exposed to a thermal stress environment and the measured baseline spontaneous blood volume oscillations at a time when user is not exposed to a thermal stress environment for the system shown in FIG. 3;

FIG. 4B shows an example of amplitudes change as measured by the processing subsystem shown in FIG. 3 and shown in FIG. 4A compared to actual oscillation amplitudes;

FIG. 5 is a schematic l view showing in further details the primary components of the thermal stress monitoring system shown in one or more of FIGS. 1-4B; and

FIG. 6 is a flow chart showing one example of the thermal stress monitoring method.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

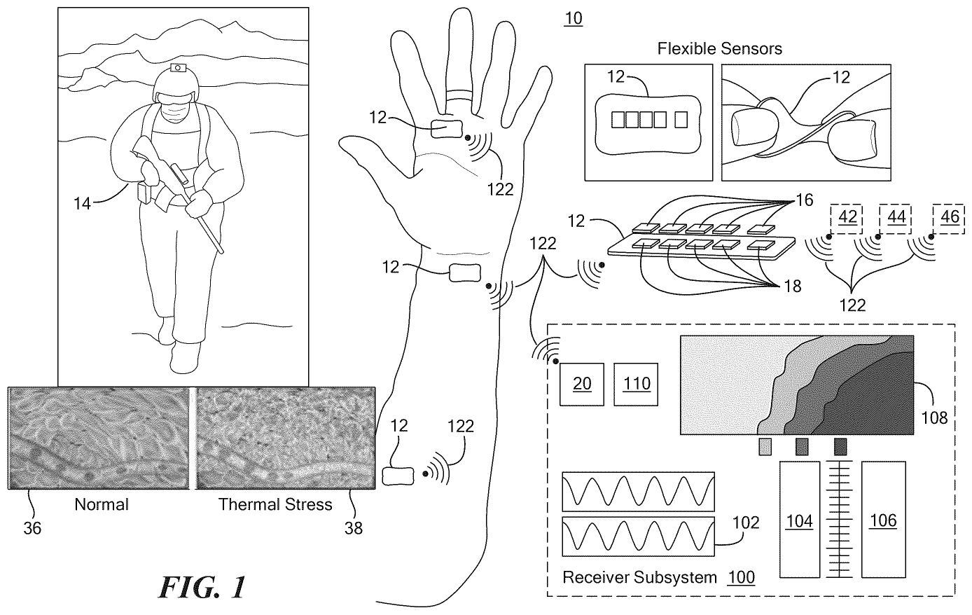

Thermal stress monitoring system 10, FIG. 1, includes at least one wearable sensor 12 adapted to be placed on a peripheral area of user 14, e.g., a hand, a finger, and/or an arm, as shown, or an ear, the nose, the face, a cheek, a leg, a foot, a toe, or similar type peripheral area of the user. At least one wearable sensor 12 sensor is sensitive to changes in spontaneous blood volume oscillations and generates output signals.

System 10 also includes processing subsystem 20 which receives the output signals and measures baseline spontaneous blood volume oscillations at a time when user 14 is not exposed to a thermal stress environment and subsequently measures exposed spontaneous blood volume oscillations at a time when user 14 is exposed to a thermal stress environment and then determines a thermal stress injury based on changes in the measured exposed blood volume oscillations from the baseline spontaneous blood volume oscillations. The determined thermal stress injury may include frostbite, hypothermia, frostnip, chilblain, immersion foot, heat strain, heat stroke, heat exhaustion, or similar type thermal stress-related injury.

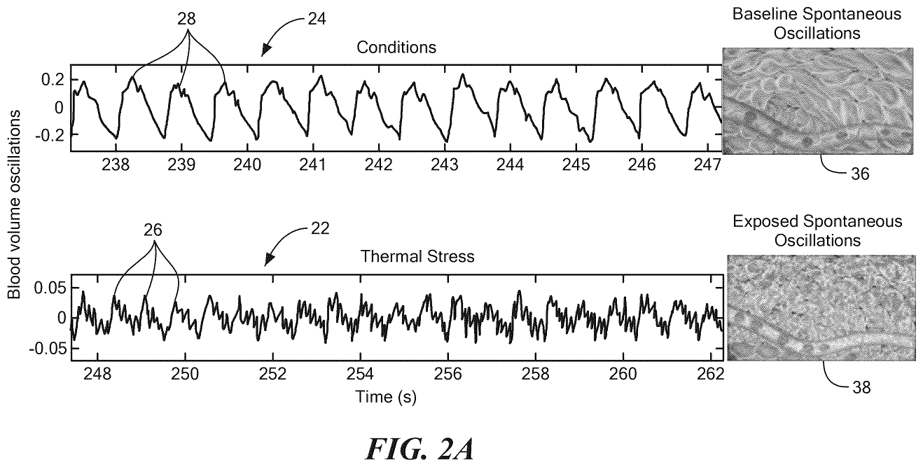

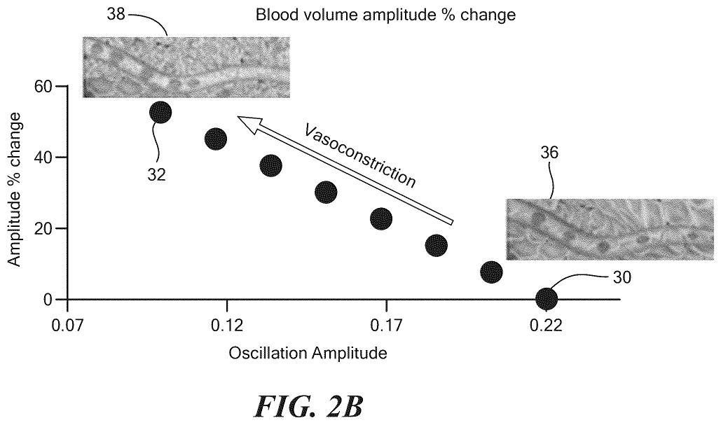

In one example, the changes in the measured exposed spontaneous blood volume oscillations from the measured baseline spontaneous blood volume oscillations may be determined evaluating the difference between the measured exposed spontaneous blood volume oscillations and the measured baseline spontaneous blood volume oscillations. For example, FIG. 2A shows an example of the measured exposed spontaneous blood volume oscillations 22 at a time when the user is exposed to a thermal stress environment and the measured baseline spontaneous blood volume oscillations 24 at a time when user 14 is not exposed to a thermal stress environment. In this example, processing subsystem 20 evaluates the difference between measured exposed spontaneous blood volume oscillations 22 and the measured baseline spontaneous blood volume oscillations 24 to determine a thermal stress injury.

In one example, the difference between measured exposed spontaneous blood volume oscillations 22 and the measured baseline spontaneous blood volume oscillations 24 to determine a thermal stress injury may include one of several comparisons that may utilize time domain differences, frequency domain differences, or both.

For example, frequency domain differences, which may include a power spectral density (PSD) or fast Fourier transform (FFT) of the output signals, may be obtained that represent the frequency domain content and the amplitude of selected frequencies in the PSD and may be summed to provide a metric that may be compared between the measured baseline spontaneous blood volume oscillations signals and the measured exposed spontaneous blood volume oscillations.

Examples of time domain difference are shown in FIG. 2A discussed above. Other examples of time domain difference may include changes in the shape and peaks shift/changes of measured exposed spontaneous blood volume oscillations 22 and measured baseline spontaneous blood volume oscillations 24, such as variations in the dicrotic and/or late systolic peak and/or pulse peak. The DC (Direct Current) component of measured exposed spontaneous blood volume oscillations 22 and the measured baseline spontaneous blood volume oscillations 24 may change as well due to overall changes in blood volume.

In another example, changes in the measured exposed spontaneous blood volume oscillations 22 from the measured baseline spontaneous blood volume oscillations 24 may be determined by evaluating the difference between amplitudes 26 of the measured exposed spontaneous blood volume oscillations 22 and amplitudes 28 of the measured baseline spontaneous blood volume oscillations 24.

FIG. 2B shows an example of the amplitudes change as measured by processing subsystem 20 compared to actual oscillation amplitudes. In this example, at point 30 there is no change in amplitudes (baseline oscillations when the user is not exposed to a thermal stress environment). At point 32, there is a large percentage of change between amplitudes 26 of exposed spontaneous blood volume oscillations 22 (when the user is exposed to thermal stress) and amplitudes 28 of baseline spontaneous blood volume oscillations 24 (when the user is not exposed to thermal stress), indicating vasoconstriction, indicated at 34, and the onset of a thermal stress injury.

Exemplarily photographs of baseline spontaneous blood volume oscillations are indicated at 36 in FIGS. 1 and 2A-2B. Exemplarily photographs of exposed spontaneous blood volume oscillations are indicated at 38.

In another example, processing subsystem 20 may determine changes in the measured exposed spontaneous blood volume oscillations 22 from baseline spontaneous blood volume oscillations 24 by evaluating a ratio between amplitudes 26 of exposed spontaneous blood volume oscillations 22 and amplitudes 28 of baseline spontaneous blood volume oscillations 24 to determine a thermal stress injury.

Processing subsystem 20 may also utilize algorithms to analyze the output signals in the frequency domain to extrapolate information about shape and frequency content changes. Additionally, processing subsystem 20 may utilize artificial intelligence or machine learning algorithms to track changes in the shape of the waveforms of measured exposed spontaneous blood volume oscillations 22 and the measured baseline spontaneous blood volume oscillations 24, such as peaks amplitude ratio changes due to a shift in the primary and secondary peak in the optical waveform, changes in the shape and peaks shift/changes of measured exposed spontaneous blood volume oscillations 22 and the measured baseline spontaneous blood volume oscillations 24, such as variations in the dicrotic and/or late systolic peak and/or pulse peak. The DC (Direct Current) component of measured exposed spontaneous blood volume oscillations 22 and measured baseline spontaneous blood volume oscillations 24 might change as well due to overall changes in blood volume.

In one design, wearable sensor 12, FIG. 1, preferably includes at least one light source 16 which emits light at one or more predetermined wavelengths, e.g., the wavelengths of green light, red light, near-infrared light, or similar type light responsive to spontaneous blood volume oscillations into the tissue of user 14. Green light may be highly absorbed by hemoglobin, making it effective for detecting blood volume changes in the microvascular bed of the skin. Red light penetrates deeper into the skin, making it useful for detecting blood flow in deeper tissue layers, and thus is less sensitive to motion artifacts. Infrared light penetrates the deepest and is less affected by skin pigmentation and ambient light, offering stable readings in varying environmental conditions.

At least one wearable sensor 12 also preferably includes at least one detector 18 which detects reflected light at one or more predetermined wavelengths associated with the spontaneous blood volume oscillations and generates output signals.

At least one light source 16 preferably emits light which penetrates the tissue of user 14 to one or more predetermined depths, e.g., about 1 mm to about 3 or 4 mm, or other desired deeper depths as needed. In one example, to detect light that is reflected or scattered from a particular depth, at least one detector 18 is preferably configured and positioned with respect to at least one light source 16 to be sensitive to light from the one or more desired predetermined depths.

At least one wearable sensor 12 may include at least one photoplethysmography (PPG) sensor 42, at least one bioimpedance analysis (BIA) sensor 44, and/or at least one ballistocardiography (BCG) sensor 46.

System 10 preferably provides early detection and management of thermal strain under challenging environmental and working conditions. To facilitate this, at least one wearable sensor 12 operating at predetermined wavelengths preferably measures the vasodilation of microvasculature, preferably using the metric of cutaneous vascular conductance (CVC). The use of optical-based measurements, such as PPG, to detect thermal strain and dynamically manage cooling interventions is scientifically validated through numerous studies. These studies demonstrate PPG's efficacy in monitoring skin blood flow changes in real-time in a non-invasive manner. PPG, which measures light absorption variations in the skin to infer blood volume changes, is preferably particularly suited for tracking physiological responses to thermal stress. Key research has established the reliability of PPG of at least one PPG sensor 42 and sensitivity across various thermal and physiological conditions. System 10 may utilize multi-site PPG sensors placed at strategically chosen anatomical sites, including the forearm, neck, and chest, and the like as discussed above to measure localized skin blood flow. These PPG sensors preferably track amplitude variations in the PPG signal, enabling real-time determination of the user's position on the CVC curve.

At least one BIA sensor 44 preferably detects changes in bioimpedance in the human subject and outputs BIA signals. At least one BIA sensor 44 is preferably less sensitive to minor changes in skin contact than at one PPG sensors 42 and preferably provides a stable signal. While the BIA signal can be affected by other factors, such as electrode placement, tissue composition, hydration levels, and the like, combining at least one BIA sensor 44 and at one PPG sensor 42 provides a single device of system 10 which preferably leverages the strengths of both methods, such as providing a stable baseline measurement with BIA and high-resolution data on blood volume changes with PPG.

At least one BCG sensor 46 preferably includes one or more accelerometers and/or gyroscopes (not shown) which preferably detect and compensate for motion and outputs BCG signals. At least one BCG sensor 46 also preferably measures micro-movements of the human subject which may be caused by the ejection of blood with each heartbeat.

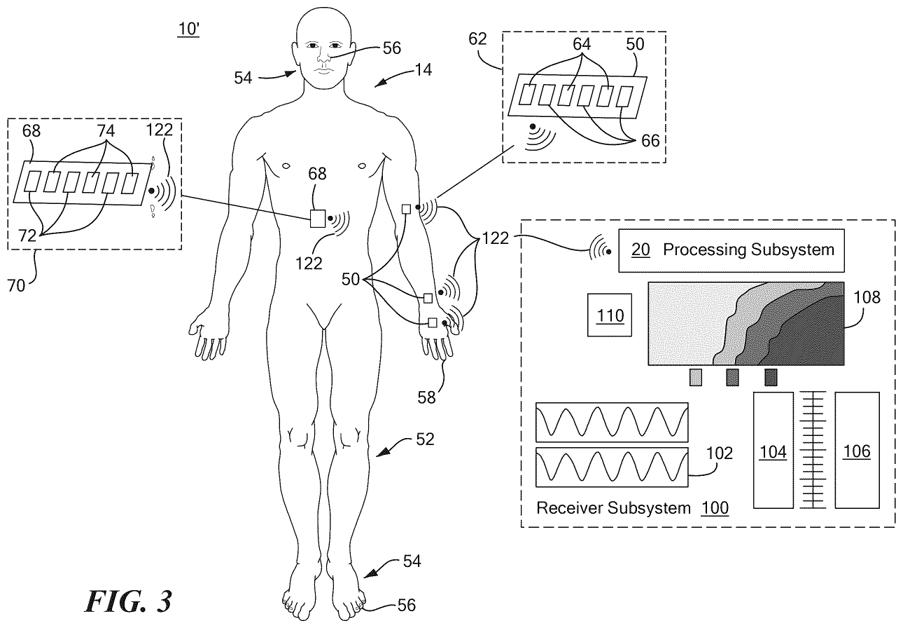

In another embodiment, system 10′, FIG. 3, where like parts have like numbers, and the method thereof preferably includes at least one peripheral wearable sensor 50, similar to wearable sensor 12, FIG. 1, adapted to be placed on a peripheral area of user 14, e.g., a hand, a finger, and/or an arm, as shown, or leg 52, foot 54, toe 56, face 54, nose 56, fingertip 58, or similar type peripheral area. Similar as discussed above, at least one peripheral wearable sensor 50, shown in greater detail in caption 62, includes at least one peripheral light source 64 which preferably emits light at one or more predetermined wavelengths. e.g., green light, red light, near-infrared light, or similar type light, responsive to spontaneous blood volume oscillations into tissue of user 14. At least one peripheral wearable sensor 50 also includes at least one peripheral detector 66 which detects reflected light at one or more predetermined wavelengths associated with the spontaneous blood volume oscillations and generates peripheral output signals.

System 10′ also includes at least one baseline wearable sensor 68 adapted to be placed on a core area of user 14, e.g., the torso as shown, or similar type core area of human subject 14. The core area of user 14 is preferably a location less affected by temperature changes than the periphery of user 14. At least one wearable sensor 68 is preferably flexible, compact, durable, and small and does not need to be wrapped around a body part of user 14.

At least one baseline sensor wearable sensor 68, shown in greater detail in caption 70, includes at least one baseline light source 72 which emits light at one or more predetermined wavelengths e.g., green light, near-infrared light, or similar type light, responsive to with spontaneous blood volume oscillations into tissue of human subject 14. At least one baseline wearable sensor 68 also includes at least one baseline detector 74 which detects reflected light at one or more predetermined wavelengths associated with the spontaneous blood volume oscillations and output baseline output signals.

System 10′ also includes processing subsystem 20 which receives the baseline output signals and measures baseline spontaneous blood volume oscillations at the core area of user 14. Processing subsystem also receives the peripheral output signals and measures the peripheral spontaneous blood volume oscillations at a time when the user is exposed to a thermal stress environment. Processing subsystem 20 then determines a thermal stress injury based on changes in the measured peripheral blood volume oscillations from the measured baseline spontaneous blood volume oscillations. Similar as discussed above, the determined thermal stress injury may include frostbite, hypothermia, frostnip, chilblain, immersion foot, heat stoke, heat strain, heat exhaustion, or similar type thermal stress-related injury.

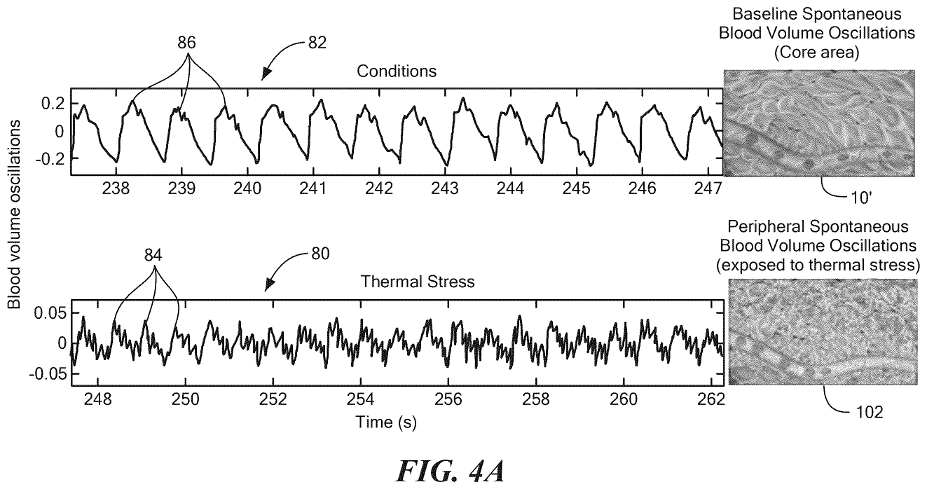

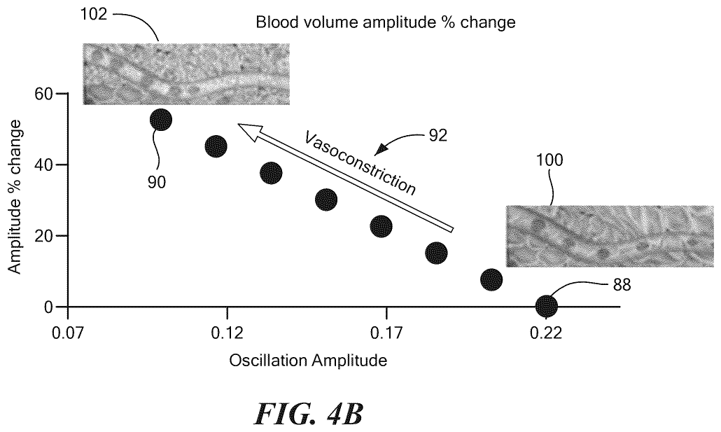

In one example, the changes in the measured peripheral spontaneous blood volume oscillations from the measured baseline spontaneous blood volume oscillations may be determined by evaluating the difference between the measured peripheral spontaneous blood volume oscillations and the measured baseline spontaneous blood volume oscillations. For example, FIG. 4A shows an example of the measured peripheral spontaneous blood volume oscillations 80 at a time when the user is exposed to a thermal stress environment and the measured baseline spontaneous blood volume oscillations 82 at the core area of user 14. In this example, processing subsystem 20 evaluates the difference between measured peripheral spontaneous blood volume oscillations 80 and measured baseline spontaneous blood volume oscillations 82 to determine a thermal stress injury.

In another example, the difference between measured peripheral spontaneous blood volume oscillations 80 and the measured baseline spontaneous blood volume oscillations 82 to determine a thermal stress injury may include one of several comparisons discussed above that may utilize time domain differences, frequency domain differences, or both.

For example, frequency domain differences, which may include a power spectral density (PSD) or fast Fourier transform (FFT) of the output signals, may be obtained that that represent the frequency domain content and the amplitude of selected frequencies in the PSD may be summed to provide a metric that is compared between the measured baseline spontaneous blood volume oscillations signals and the measured peripheral spontaneous blood volume oscillations.

Examples of time domain difference are shown in FIG. 4A discussed above. Other examples of time domain difference may include changes in the shape and peaks shift/changes of measured peripheral spontaneous blood volume oscillations 80 and measured baseline spontaneous blood volume oscillations 82, such as variations in the dicrotic and/or late systolic peak and/or pulse peak. The DC (Direct Current) component of measured peripheral spontaneous blood volume oscillations 80 and the measured baseline spontaneous blood volume oscillations 82 might change as well due to overall changes in blood volume

Similar as discussed above with reference to FIGS. 2A and 2B, in one example, the changes in the measured peripheral spontaneous blood volume oscillations 80, FIG. 4A, from the measured baseline spontaneous blood volume oscillations 82 may be determined by evaluating the difference between amplitudes 84 of the measured exposed spontaneous blood volume oscillations 80 and amplitudes 86 of the measured baseline spontaneous blood volume oscillations 82.

FIG. 4A shows an example of baseline spontaneous blood volume oscillations 82 when at least one baseline wearable sensor 68 is placed on a core area of user 14, e.g., as shown in FIG. 3, and peripheral spontaneous blood volume oscillations 80 at a time when the user is exposed to a thermal stress environment. Processing subsystem 20 preferably evaluates the difference between the amplitudes 84 of peripheral spontaneous blood volume oscillations 82 and the amplitudes 86 of baseline spontaneous blood volume oscillations 80 to determine a thermal stress injury. FIG. 4B shows an example of the amplitudes change as measured by processing subsystem 20 compared to actual oscillation amplitudes. In this example, at point 88 there is no change in the amplitudes (when baseline wearable sensor 68 is placed on a core area of user 14). At point 90, there is a large percentage of change between amplitudes 84 of peripheral spontaneous blood volume oscillations 80 (when the user is exposed to thermal stress) and amplitudes 86 of baseline spontaneous blood volume oscillations 82 (when baseline wearable sensor 68 is placed on a core area of user 14), indicating vasoconstriction, indicated at 92, and a thermal stress injury.

In this example, example photographs of baseline spontaneous blood volume oscillations 82 are indicated at 100 and exemplary photographs of peripheral spontaneous blood volume oscillations 80 are indicated at 102.

In another example, processing subsystem may determine the changes in the measured peripheral spontaneous blood volume oscillations 80 from baseline spontaneous blood volume oscillations 82 by evaluating a ratio between amplitudes 84 of peripheral spontaneous blood volume oscillations 80 and amplitudes 86 of baseline spontaneous blood volume oscillations 82 to determine a thermal stress injury.

Similar as discussed above with reference to FIGS. 1-2B, processing subsystem 20, FIG. 3, may also utilize algorithms to analyze the output signals in the frequency domain to extrapolate information about shape and frequency content changes. Additionally, processing subsystem 20 may utilize artificial intelligence or machine learning algorithms to track changes in the shape of the waveforms of measured peripheral spontaneous blood volume oscillations 80 and the measured baseline spontaneous blood volume oscillations 82, such as peaks amplitude ratio changes due to a shift in the primary and secondary peak in the optical waveform.

In one design, at least one wearable baseline sensor 68 preferably generates at least one reference signal to be used by processing subsystem 20 to improve the accuracy of the determined thermal stress injury. In this example, at least one reference signal preferably compensates for physiological variations of user 14 because at least one wearable baseline sensor 68 is preferably located at the core area of user 14 which is typically unaffected by vasoconstriction phenomena.

Processing subsystem 20 may include one or more processors, an application-specific integrated circuit (ASIC), firmware, hardware, and/or software (including firmware, resident software, micro-code, and the like) or a combination of both hardware and programs that may all generally be referred to herein as a “processing subsystem”, which may be part of system 10. Electronic storage device 110 may include any combination of computer-readable media or memory. The computer-readable media or memory may be a computer-readable signal medium or a computer-readable storage medium. The computer-readable storage medium or memory may be electronic, magnetic, optical, electromagnetic, infrared, a semiconductor subsystem, apparatus, or device, or any suitable combination of the foregoing. Other examples of electronic storage device 110 may include an electrical connection having one or more wires, random access memory (RAM), read-only memory (ROM), erasable programmable read-only memory (EPROM or Flash memory), optical fiber, an optical storage device, a magnetic storage device, or any suitable combination of the foregoing. As disclosed herein, electronic storage device 110 may be any tangible medium that can contain, or store one or more programs for use by or in connection with one or more processors of processing subsystem 20. Processing subsystem 20 and electronic storage device 110 may be implemented in a single ASIC or as a combination of integrated circuits, each implementing one of more or the subsystem functions. Processing subsystem 20 may also include wireless communications capability to transmit data to a remote processing or display system.

Computer program code for the one or more programs for carrying out the instructions or operation of one or more embodiments system 10 may be written in any combination of one or more programming languages, including an object-oriented programming language, e.g., C++, Smalltalk, Java, and the like, and conventional procedural programming languages, such as the “C” programming language, Assembly language or similar programming languages.

In other designs, system 10 shown in one or more of FIGS. 1A-4B may include at least two wearable sensors adapted to be placed on two different predetermined areas of user 14, e.g., at least one wearable peripheral sensor 50, FIG. 3, and at least one baseline wearable sensor 68, and processing subsystem 20 measures pulse wave velocity (PWV) to determine change in blood flow (between the peripheral spontaneous blood volume oscillations and the baseline spontaneous blood volume oscillations) correlated to vasoconstriction or vasodilation. Vasoconstriction and vasodilation directly affect the way the pulse wave propagates in the blood vessels. Therefore, vasoconstriction and vasodilation may be estimated by directly measuring PWV. The same holds true for blood flow changes.

In another example, system 10, FIG. 1, may detect at least one condition that restricts perfusion of peripheral tissue of user 14. In this example, processing subsystem 20 receives the output signals as similar as discussed above with reference to FIG. 1 and measures baseline spontaneous blood volume oscillations at a time when the user does not have a condition that restricts perfusion of peripheral tissues. Processing subsystem 20 subsequently measures conditional spontaneous blood volume oscillations at a time when the user has at least one condition that restricts perfusion of peripheral tissue and determines at least one condition that restricts perfusion of peripheral tissue based on changes in the measured conditional spontaneous blood volume oscillations from the baseline spontaneous blood volume oscillations. The at least one condition that restricts perfusion of peripheral tissue may include peripheral vascular disease, similar type diseases, or any condition that may impact or alter the regular and health flow of blood. In this example, at least one wearable sensor 12 may be adapted to be placed on at least one of a hand, a finger, an arm, a leg, a foot, a toe, a face, an ear, a nose, or a core area of the user.

Similar as discussed above, in one example, the changes in the measured conditional spontaneous blood volume oscillations from the measured baseline spontaneous blood volume oscillations may be determined evaluating the difference between the measured conditional spontaneous blood volume oscillations and the measured baseline spontaneous blood volume oscillations. The difference between the measured conditional spontaneous blood volume oscillations and the measured baseline spontaneous blood volume oscillations to determine at least one condition that restricts perfusion of peripheral tissue may include one of several comparisons that may utilize time domain differences, frequence domain differences, or both, as discussed above.

In one design, processing subsystem 20, FIG. 1 is preferably located remotely from at least one wearable sensor 12, FIG. 1. Similarly, processing subsystem 20, FIG. 3, is preferably located remotely from at least one peripheral wearable sensor 50 and at least one baseline wearable sensor 68. Processing subsystem 20, FIG. 1, preferably receives the output signals from wearable sensor 12 wirelessly, indicated at 122, continuously, and in real-time. Similarly processing subsystem 20, FIG. 3, preferably receives the peripheral output signals and the baseline output signals from at least one peripheral wearable sensor 50 and at least one base line sensor 68, respectfully, wirelessly, indicated at 122, continuously, and in real-time. Processing subsystem 20 preferably receives the BIA signals and BCG signals wirelessly, indicated at 122, continuously, and preferably in real-time.

System 10. FIGS. 1 and 3, may include receiver subsystem 100 which preferably includes processing subsystem 20 as discussed above. Receiver subsystem 100 preferably includes one or more displays, e.g., displays 102, 104, 106, and 108 which preferably display optical signals associated with the spontaneous blood volume oscillations discussed above, e.g., vasodilation, vasoconstriction, and temperature, respectively, and the like. The one or more displays may provide critical information to medical personnel as to the state of thermal injury of the user. The Information may include an indication of the onset of a thermal stress injury, PWV to estimate blood flow, and vasoconstriction and vasodilation percent changes, which can be displayed and stored, e.g., in electronic storage device 110.

The result is thermal stress injury detection system 10, shown in one or more of FIGS. 1A-4B, efficiently and effectively determines a thermal stress injury, such as frostbite, hypothermia, frostnip, chilblains, immersion foot, heat stroke, heat strain, heat exhaustion, or similar type thermal stress-related injuries. System 10 preferably determines an onset of a thermal stress injury prior to any tissue destruction. System 10 is preferably lightweight, portable, and may be comfortably worn by user 14 without posing any risk or impediment to movement. System 10 and the method thereof can also efficiently and effectively determine at least one condition that restricts perfusion of peripheral tissue.

At least one wearable sensor 12, FIG. 1 and/or at least one peripheral wearable sensor 50, FIG. 3, and/or at least one baseline wearable sensor 68 are each preferably easy to apply and position on the skin of user 14 as shown and can automatically detect hazard conditions while alerting the user and conveying information to medical (or non-medical) personnel.

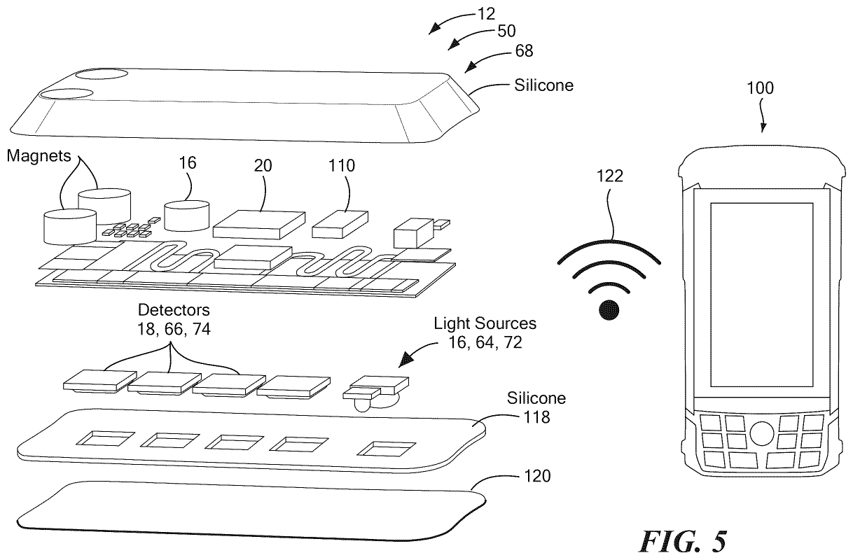

FIG. 5 shows an exploded detail view of one example of at least one wearable sensor 12, FIG. 1, at least one peripheral wearable sensor 50, FIG. 3, and at least one baseline wearable sensor 68. In one design each of wearable sensors 12, 50, and 68 preferably include flexible circuit board 112, electronic components 114, e.g., processing subsystem 20, electronic storage device 110, power supply 116, and the like, at least one light source 16, 64, 72, FIGS. 1 and 3, e.g., a light emitting diode (LED), or similar type light source, and at least one detector 18, 66, 74, e.g., a photodiode, an avalanche photodiode, photomultiplier tubes, a charge-coupled device (CCD), a complementary metal-oxide-semiconductor (CMOS) sensor, or similar type detector. Each of wearable sensors 12, 50, and 68 also preferably include flexible cover 118 made of a flexible material such as silicone or similar type material, flexible base 120 made of a flexible material such as silicone or similar type material, and adhesive layer 120 for attaching wearable sensors 12, 50, and 68 to user 14.

In this example, receiver subsystem 100 is preferably configured as a smart device as shown and is preferably located remotely from wearable sensors 12, 50, and 68 and receives the output signal, peripheral output signals, and the baseline output signals wirelessly, indicated at 122, continuously, and in real-time as shown.

Thermal stress injuries typically result from inadequate protection against a thermal stress environment. For example, frostbite may result from inadequate protection against the cold and most frequently affects the hands and feet but can also occur on ears, nose, face, cheeks, fingers, toe, and the like. Frostbite can be categorized as a localized freezing thermal stress injury. Skin does not freeze until it cools to about −4° C. or colder. Wind increases the rate at which skin cools but does not affect the temperature at which skin freezes. In response, the U.S. Armed Forces have developed and improved training, doctrine, procedures, and protective equipment and clothing to counter the threat of thermal stress environments. Although these measures are highly effective, cold injuries have continued to affect hundreds of military personnel, winter and outdoor sports athletes, construction and utility workers, homeless or displaced persons, and the like, each year because of exposure to cold and wet environments. Frostbite occurs in four interconnected progressive pathophysiologic phases that are dependent on the rate and duration of freezing, rate of rewarming, and anatomic extent of exposure. The initial response to skin cooling is vasoconstriction, which helps defend the core temperature against loss of heat via the skin and translates to smaller blood volume oscillations as shown by preliminary data. FIGS. 2A-2B and 4A-4B, discussed above, show an examples of prototype-acquired data which show the effectiveness of system 10 and the method thereof for detecting the onset of frostbite.

In the extremities, this is followed by cold-induced vasodilation (CIVD), also known as the “hunting response”, which protects against thermal stress injury at the cost of increased heat loss. As the skin cools further, blood viscosity increases, and there is microvasculature constriction with transendothelial plasma leakage. Because of the sensitivity and specificity of processing subsystem 20, FIGS. 1 and 3, in detecting vasoconstriction and vasodilation changes output by at least one wearable sensor 12, FIG. 1, and/or at least one peripheral wearable sensor 50, FIG. 3, and/or at least one baseline wearable sensor 68, system 10 preferably measures the initial body responses to skin cooling before hypoxia and acidosis damage the endothelium and coagulation takes place. By properly choosing predetermined range of wavelengths output by at least one wearable sensor 12, FIG. 1 and/or at least one peripheral wearable sensor 50, FIG. 3, and/or at least one baseline wearable sensor 68, system 10 preferably maximizes the sensitivity of the optical measurements to the change in chromophores in blood, e.g., to use this information to measure baseline spontaneous blood volume oscillations 22, FIG. 2A, exposed spontaneous blood volume oscillations 24, baseline spontaneous blood volume oscillations 80, FIG. 4A and peripheral spontaneous blood volume oscillations 82 and to relate those blood volume oscillations to vasoconstriction and vasodilation changes, e.g., as shown in FIGS. 2B and 4B. However, the absolute amplitude values of blood volume oscillations naturally vary among different individuals. Thus, system 10 preferably utilizes a short calibration period, e.g., less than about 10 s, for each individual user the first time system 10 is used before exposure to cold so that system 10 can continuously monitor relative percentage blood volume oscillation changes with respect to the baseline amplitude values determined during the calibration procedure. Skin blood flow is under both central and local control. In cold conditions, vasoconstriction is the initial response of the body and baseline values of blood flow decrease by about 10 percent. This effect is not enough to cause ischemia, but it does indicate that the thermoregulatory functions of the skin have been activated, and these incremental percent losses correlate to the onset of frostbite. Once maximal vasoconstriction has occurred, vasodilation will happen (CIVD). In one design, system 10 preferably includes multiple optical sensors, e.g., at least one wearable sensor 12 as shown in FIG. 1 or at least one peripheral wearable sensor 50 and at least one baseline wearable sensor 68 as shown in FIG. 3, which preferably emit light at one or more predetermined wavelengths associated with spontaneous blood volume oscillations into tissue of user 14 as discussed above and preferably at multiple skin depths as discussed above to measure blood volume oscillations at multiple tissue layers. By allowing system 10 to measure vasoconstriction and vasodilation in all layers, system 10 can distinguish frostbite even in severe cold conditions when the hemodynamics of the outer surface of the skin might be compromised but cold has not penetrated deeply enough into the tissue to cause frostbite.

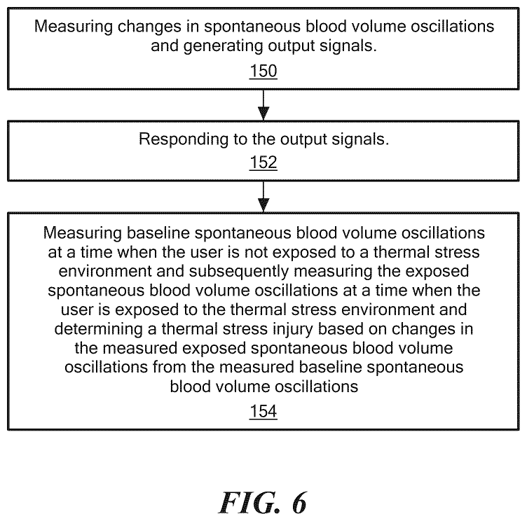

One example of the thermal stress monitoring method includes measuring changes in spontaneous blood volume oscillations and generating output signals, step 150, FIG. 6, responding to the output signals, step 152, and measuring baseline spontaneous blood volume oscillations at a time when the user is not exposed to a thermal stress environment and subsequently measuring the exposed spontaneous blood volume oscillations at a time when the user is exposed to the thermal stress environment and determining a thermal stress injury based on changes in the measured exposed spontaneous blood volume oscillations from the measured baseline spontaneous blood volume oscillations, step 154.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A thermal stress monitoring system, the system comprising:
at least one wearable sensor adapted to be placed on a human subject, the at least one wearable sensor sensitive to changes in spontaneous blood volume oscillations and configured to generate output signals; and
a processing subsystem configured to receive the output signals and configured to measure baseline spontaneous blood volume oscillations at a time when the user is not exposed to a thermal stress environment and subsequently measure the exposed spontaneous blood volume oscillations at a time when the user is exposed to the thermal stress environment and determine a thermal stress injury based on changes in the measured exposed spontaneous blood volume oscillations from the measured baseline spontaneous blood volume oscillations.

2. The system of claim 1 in which the changes in the measured exposed spontaneous blood volume oscillations from the measured baseline spontaneous blood volume oscillations are determined by evaluating the difference between the measured exposed spontaneous blood volume oscillations and the measured baseline spontaneous blood volume oscillations.

3. The system of claim 2 in which the difference between the measured exposed spontaneous blood volume oscillations and the measured baseline spontaneous blood volume oscillations includes one or more comparisons that utilize time domain differences, frequency domain differences, or both.

4. The system of claim 1 in which the at least one wearable sensor includes at least one photoplethysmography (PPG) sensor, at least one bioimpedance analysis (BIA) sensor, and/or at least one ballistocardiography (BCG) sensor.

5. The system of claim 1 in which the at least one wearable sensor includes at least one light source configured to emit light at one or more predetermined wavelengths associated with spontaneous blood volume oscillations into tissue of the human subject and at least one detector configured to detect reflected light at the one or more predetermined wavelengths associated with the spontaneous blood volume oscillations and generate the output signals.

6. A thermal stress monitoring system, the system comprising:
at least one peripheral wearable sensor adapted to be placed on a peripheral area of a user, the peripheral wearable sensor including:
at least one peripheral light source configured to emit light at one or more predetermined wavelengths associated with spontaneous blood volume oscillations into tissue of a human subject, and
at least one peripheral detector configured to detect reflected light at the one or more predetermined wavelengths associated with the spontaneous blood volume oscillations and generate peripheral output signals;
at least one baseline wearable sensor adapted to be placed on a core area of a user, the baseline wearable sensor including:
at least one baseline light source configured to emit light at one or more predetermined wavelengths associated with spontaneous blood volume oscillations into tissue of a human subject, and
at least one baseline detector configured to detect reflected light at the one or more predetermined wavelengths associated with the spontaneous blood volume oscillations and generate baseline output signals; and
a processing subsystem configured to receive the baseline output signals and configured to measure the baseline spontaneous blood volume oscillations and configured to receive the peripheral output signals and measure the peripheral spontaneous blood volume oscillations at a time when the user is exposed to a thermal stress environment and determine a thermal stress injury based on changes in the measured peripheral blood volume oscillations from the measured baseline spontaneous blood volume oscillations.

7. The system of claim 6 in which the changes in the measured peripheral spontaneous blood volume oscillations from the measured baseline spontaneous blood volume oscillations are determined by evaluating the difference between the measured peripheral spontaneous blood volume oscillations and the measured baseline spontaneous blood volume oscillations.

8. The system of claim 7 in which the difference between the measured peripheral spontaneous blood volume oscillations and the measured baseline spontaneous blood volume oscillations includes one or more comparisons that utilize time domain differences, frequency domain differences, or both.

9. The system of claim 7 in which the at least one wearable baseline sensor is adapted to be placed on a core area of the user.

10. The system of claim 6 in which the at least one wearable baseline sensor generates at least one reference signal to be used by the processing subsystem to improve the accuracy of the determined thermal stress injury.

11. A system for detecting at least one condition that restricts perfusion of peripheral tissue, the system comprising:
at least one wearable sensor adapted to be placed on a human subject, the at least one wearable sensor sensitive to changes in spontaneous blood volume oscillations and configured to generate output signals; and
a processing subsystem configured to receive the output signals and configured to measure baseline spontaneous blood volume oscillations at a time when the user does not have at least one condition that restricts perfusion of peripheral tissue and subsequently measure conditional spontaneous blood volume oscillations at a time when the user has at least one condition that restricts perfusion of peripheral tissue and determine at least one condition that restricts perfusion of peripheral tissue based on changes in the measured conditional spontaneous blood volume oscillations from the baseline spontaneous blood volume oscillations.

12. The system of claim 11 in which the changes in the measured conditional spontaneous blood volume oscillations from the measured baseline spontaneous blood volume oscillations is determined by evaluating the difference between the measured conditional spontaneous blood volume oscillations and the measured baseline spontaneous blood volume oscillations.

13. The system of claim 11 in which the difference between measured conditional spontaneous blood volume oscillations and the measured baseline spontaneous blood volume oscillations includes one or more comparisons that utilize time domain differences, frequence domain differences, or both.

14. The system of claim 11 in which the at least one wearable sensor includes photoplethysmography (PPG) sensor, at least one bioimpedance analysis (BIA) sensor, and/or at least one ballistocardiography (BCG) sensor.

15. The system of claim 11 in which the at least one wearable sensor includes at least one light source configured to emit light at one or more predetermined wavelengths associated with spontaneous blood volume oscillations into tissue of the human subject and at least one detector configured to detect reflected light at the one or more predetermined wavelengths associated with the spontaneous blood volume oscillations and generate the output signals.

16. A thermal stress monitoring method, the method comprising:
measuring changes in spontaneous blood volume oscillations and generating output signals;
responding to the output signals; and
measuring baseline spontaneous blood volume oscillations at a time when the user is not exposed to a thermal stress environment and subsequently measuring the exposed spontaneous blood volume oscillations at a time when the user is exposed to the thermal stress environment and determining a thermal stress injury based on changes in the measured exposed spontaneous blood volume oscillations from the measured baseline spontaneous blood volume oscillations.

17. The method of claim 16 in which the changes in the measured exposed spontaneous blood volume oscillations from the measured baseline spontaneous blood volume oscillations are determined by evaluating the difference between the measured exposed spontaneous blood volume oscillations and the measured baseline spontaneous blood volume oscillations.

18. The method of claim 17 in which the difference between the measured exposed spontaneous blood volume oscillations and the measured baseline spontaneous blood volume oscillations includes one or more comparisons that utilize time domain differences, frequency domain differences, or both.

19. The method of claim 16 including emitting light at one or more predetermined wavelengths associated with spontaneous blood volume oscillations into tissue of the human subject and detecting reflected light at the one or more predetermined wavelengths associated with the spontaneous blood volume oscillations and generating the output signals.
